(19) United States
(12) Patent Application Publication
(10) Pub. No.: US 2017/0087324 A1
(43) Pub. Date: Mar. 30, 2017

(54) CO-EXTRUDED HOSE WITH SYMMETRY PROPERTIES

(71) Applicant: inSleep Technologies, LLC, Weston, FL (US)

(72) Inventors: Martin W. Kerber, Eden Praire, MN (US); Wayne D. Robens, Eden Praire, MN (US); Craig L. Peterson, Carver, MN (US)

(21) Appl. No.: 14/866,516

(22) Filed: Sep. 25, 2015

(51) Int. Cl. A61M 16/08; F16L 11/22; A61M 16/06

(52) U.S. Cl. CPC A61M 16/0875; A61M 16/0666; F16L 11/22; A61M 2205/02

(57) ABSTRACT

A hose having an asymmetrical cross-section but which exhibits symmetrical bending and crush resistance regardless of the direction of the bending and/or crushing forces exerted on the tube. The tube includes an outer wall and an inner strengthening rib which is coextruded with the outer wall, the inner rib fabricated from a material having a durometer which is at least equal to, or higher, i.e., equal or higher resistance to deflection and/or indentation, than the material out of which the outer wall is fabricated.

### CROSS REFERENCE TO RELATED APPLICATIONS

This application is a continuation-in-part of and claims priority to U.S. patent application Ser. No. 14/702,135 filed May 1, 2015 entitled “Co-Extruded Hose with Symmetry Properties;” is a continuation-in-part of and claims priority to U.S. patent application Ser. No. 29/522,917 filed Apr. 3, 2015 entitled “Breathing Tube;” is a continuation-in-part of and claims priority to U.S. patent application Ser. No. 13/289,178 filed Nov. 4, 2011 entitled “Breathing Apparatus,” which itself claims priority to U.S. Provisional Patent Application Ser. No. 61/410,134 filed Nov. 4, 2010 and U.S. Provisional Patent Application Ser. No. 61/423,195 filed Dec. 15, 2010; is a continuation-in-part of U.S. patent application Ser. No. 13/425,049 filed Mar. 20, 2012 entitled “Breathing Apparatus,” which itself claims priority to U.S. Provisional Patent Application Ser. No. 61/467,760 filed Mar. 25, 2011; and is a continuation-in-part of and claims priority to Ser. No. 13/534,984 filed Jun. 27, 2012 entitled “Replaceable Nasal Interface System,” which itself claims priority to U.S. Provisional Patent Application Ser. No. 61/501,444 filed Jun. 27, 2011, the contents of which are hereby incorporated by reference in their entirety.

### BACKGROUND OF THE INVENTION

1. Field of the Invention

This invention relates to the field of fluid carrying tubes and hoses, and more particularly relates to tubes and hoses that are asymmetrical and crush/crimp-resistant, yet exhibit symmetrical resistance to crimping/crushing and bending.

2. Description of the Prior Art

Tubes having strengthening features are known in the art. Many different approaches have been taken to eradicate or lessen the effects of external forces exerted on hoses. For example, helically wrapped rigid structures (e.g. wire) have been used, as have two or more constituent materials having differing hardness or flexibility, otherwise known as the materials' durometer. Stiffening ribs have also been used to provide structural integrity to a tube to reduce the effects of external forces on the constituent fluids within a tube.

Tubes allowing for the flow of a plurality of fluids in a single tube have also been proposed in the past. For example, hemodialysis catheters are routinely provided with two or more passageways or lumens to permit the removal of blood from the patient's body, transportation to a dialysis machine, and return of the treated blood to the body through a second lumen within the same tube.

Numerous attempts have been made in the prior art to optimize the multi-lumen configuration.

In some approaches, such as disclosed in U.S. Pat. Nos. 4,568,329 and 5,053,023, the inflow and outflow lumen themselves are provided side by side in D-shaped form. In other approaches, such as those disclosed in U.S. Pat. Nos. 4,493,696, 5,167,623 and 5,380,276, the inflow and outflow tubes are placed in concentric relation. Other examples of different lumen configurations are disclosed in U.S. Pat. Nos. 5,221,256, 5,364,344, and 5,451,206. U.S. Pat. Nos. 6,814,718 and 7,011,645 disclose other lumen configurations. However, there remains an unmet need for tubes which themselves have an asymmetrical cross-sectional profile, for which the current art does not provide.

Many forms of respiratory therapy involve use of a respirator hose to convey a breathing gas such as air to a patient. For such use, the respirator hose must be light enough to move about easily, but still be strong enough to resist crushing or kinking that would close off flow of the breathing gas. A common practice for strengthening such respirator hoses is to wrap a thin, flexible plastic membrane about a helical or spiral support structure. The spiral support structure is typically formed of a hard metal wire or plastic.

In the provision of pressurized breathing gas provided to patients in the administration of CPAP therapy, it is necessary to convey breathable gas from a source of pressurized breathing gas to a breathing interface worn by the patient. In instances where it is desirable to detect the pressure at the interface and feed that pressure back to a sensor associated with the air supply, a pressure sensing lumen may be used. The simplest way to incorporate a pressure sensing lumen with a breathable gas supply tube is to connect the pressure sensing lumen to the gas supply tube. One way to accomplish this is to provide a multi-lumen tube.

However, a pressure sensing lumen is not required for the CPAP therapy system to work. In many CPAP devices, pressure is sensed not at the user breathing interface, but at or near the blower apparatus. Thus, adding an extra lumen to the tube as a pressure sensor would be unnecessary.

In as much as the breathing gas tube or tubes employed with some CPAP systems make contact with the wearer's face, it is critical to reduce to the greatest extent possible any discomfort experienced by the wearer in areas where the tube contacts the face. One means of doing so is to form the tube with a somewhat flattened skin-facing surface, such as would be realized with a D-shaped cross-sectional profile. Such a configuration has the effect of increasing the contact area between the tube and the skin, which in turn reduces the discomfort perceived by the wearer.

Users of CPAP systems also face the challenges associated with movements during sleep. Such movements create crushing and kinking forces on the tube, which can interfere with, and even completely interrupt, the flow of breathing gas to the wearer. Therefore, it is critical that a tube be as strong as possible, yet light weight enough to be as unobtrusive as possible. These competing needs create an especially difficult challenge in the case of D-shaped breathing gas tubes, due to the asymmetrical cross-sectional profile thereof. The movements create essentially infinitely varying deflections in the tube depending upon the direction of the forces exerted upon the tube (e.g. radial, columnar, bending, etc.). Therefore, if the structural configuration and compositional material used to create the tube is not carefully chosen, the D-shaped tube will be easy to kink and/or crush.

The use of coextruded materials having different durometers is known. For example, U.S. Pat. No. 5,451,206 to Young discloses a triple lumen catheter formed from coextrusion using materials of different durometers, in which an inner septum or dividing wall is formed using a material having a durometer greater than the material which is used to form the outer cylindrical tube. However, not only does the tube of Young have a cylindrical cross-sectional profile, but the third, smaller, lumen, along with the two larger lumen, are bounded in part by the softer outer peripheral wall of the tube. This creates a situation where pressure forces in one lumen may collapse any of the lumens due to the softer wall material used to form each of the lumen. U.S. Pat. No. 5,221,256 to Mahurkar discloses a multi-lumen catheter of cylindrical cross-sectional shape having an internal diametral septum extending along the length thereof. The third lumen of the Mahurkar tube is also bounded by the lower durometer material, allowing for crushing and/or kinking In addition, a rigid septum is more complicated and expensive to incorporate during the extrusion process than simply coextruding a tube out of a single base material, but having differing durometers in the different components of the tube.

What is needed, therefore, is a hose that has substantially equal bending resistance in all directions to reduce the tendency of the hose to crimp.

Therefore, it is a principal object of this invention to provide a tube of the type having an asymmetrical cross-sectional profile/area but which exhibits symmetrical bending and crush resistance, i.e., uniform resistance to bending and/or crushing, regardless of the direction of the bending and/or crushing forces exerted on the tube.

It is also an object of the invention to provide a fluid flow tube, regardless of the number of lumen which it defines, which has an asymmetrical cross-sectional profile/area but which exhibits symmetrical bending and crush resistance.

It is a further object of one embodiment of the invention to provide a flexible fluid flow tube which has a somewhat, or alternatively completely, flattened side adapted to rest against a user's face or other body part which demonstrates omnidirectional rigidity against crushing and kinking forces.

These and other objects are achieved by the configuration and arrangement of component parts of a tube as shown and described herein.

### SUMMARY OF THE INVENTION

The present invention provides a hose having an asymmetrical cross-section but which exhibits symmetrical bending and crush resistance regardless of the direction of the bending and/or crushing forces exerted on the tube. Such a tube may have an inner strengthening rib which is coextruded with the outer wall of the tube. In at least one embodiment the rib defines at least one additional lumen, and is fabricated of a material having an equal or higher durometer, i.e., equal or higher resistance to deflection and/or indentation than the material out of which the outer wall is fabricated. This arrangement provides a flexible tube body but which will resist kinking and crushing to the greatest extent possible.

### BRIEF DESCRIPTION OF THE DRAWINGS

FIG. 1 is a cross-sectional elevational view of an embodiment of the invention.

FIG. 2 is a side elevational view of a section of tube made in accordance with this invention.

FIG. 3 is an enlargement of the area of detail shown in FIG. 1.

FIG. 4 is a perspective view of a section of hose made in accordance with the invention.

FIG. 5 is a perspective view of a section of hose made in accordance with the invention incorporated within a breathing supply system worn by a patient.

FIG. 6 is a top perspective view of a section of hose made in accordance with the invention in proximity to a connector of the breathing supply system.

FIG. 7 is a cross-sectional elevational view of a second embodiment of the invention.

FIG. 8 is a side elevational view of a section of tube made in accordance with this invention.

FIG. 9 is an enlargement of the area of detail shown in FIG. 7.

FIG. 10 is a perspective view of a section of hose made in accordance with the invention.

FIG. 11 is a perspective view of a section of hose made in accordance with the invention incorporated within a breathing supply system worn by a patient.

FIG. 12 is a top perspective view of a section of hose made in accordance with the invention in proximity to a connector of the breathing supply system.

### DESCRIPTION OF THE PREFERRED EMBODIMENT

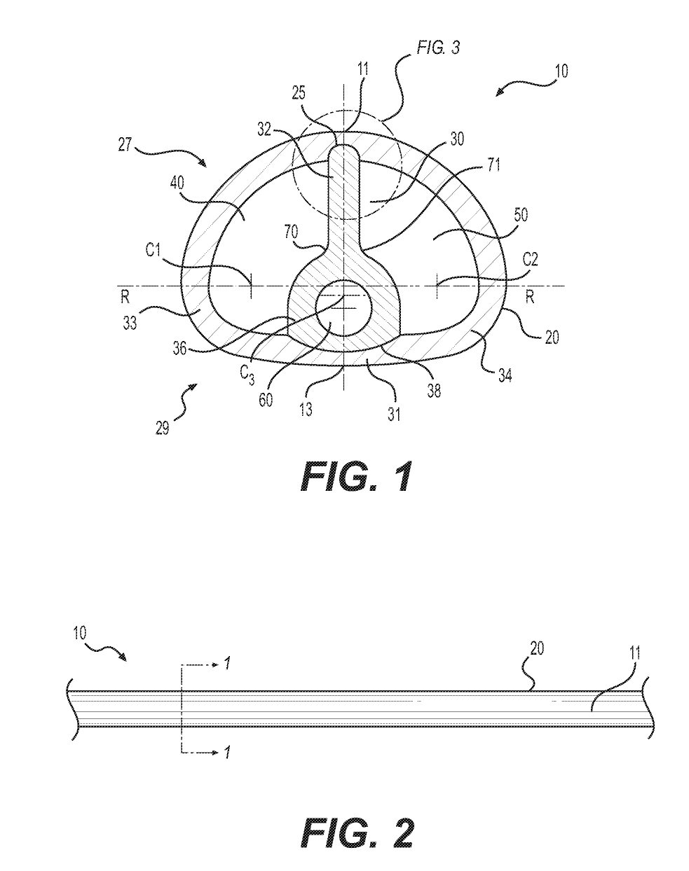

This invention is suited to, among many other things, application to tubes used to supply a respirable breathing gas under pressure from a pressure source to a respiratory interface worn by a user. In the preferred embodiment, the breathable gas is adapted to be supplied under pressure at or below the range used for the treatment of sleep disordered breathing. Such pressurized gas creates a pneumatic stent of the upper respiratory tract of a patient, whereby obstructions which may cause snoring, apnea or the like are displaced out of the breathing space. Such apparatus, known as CPAP (continuous positive airway pressure) devices, deliver breathable gas at a predetermined, substantially constant, pressure to achieve the aforementioned stenting. A suitable gas supply apparatus S, as seen in FIG. 1, for use with hoses of this invention is disclosed in commonly owned U.S. patent application Ser. No. 13/425,049, the entire contents of which are hereby incorporated by reference as though fully set forth herein. In that apparatus, a control system is configured to provide a control signal to the blower for controlling the pressure of the supply of breathing gas at the user breathing interface. The control signal may be based upon, at least in part, the pressure of the supply of breathing gas at the user breathing interface, or may otherwise be based upon, at least in part, the flow rate of the supply of breathing gas at the user breathing interface, or still further may be based upon, at least in part, a pressure within or near the mouth and/or nares of the user.

As used herein “tube” or “tubes” shall mean a hose or hoses made from an extruded material having at least one outer wall and one inner wall which allows for the free flow of a fluid, gas, or mixed phase materials within. A tube may have a plurality of inner walls forming one or more lumen within a tube. A tube may be made from one or a combination of materials selected from polyvinyl chloride (PVC), rubber, tygon, polyurethane, silicon, or any polymers, combinations or derivatives thereof. In at least one embodiment the tube materials are medical grade.

As used herein “asymmetrical” shall mean, only as to the preferred embodiment, a tube having a non-circular cross-sectional area when cut through a plane that is perpendicular to an elongate axis of the tube.

As used herein, the term “flattened” in connection with a fluid-carrying hose shall mean a planar tube wall section, or, if curved, a tube wall section having a radius of curvature that is greater than a radius of curvature of any other aspects of the tube.

The pressure sensor of such device may be coupled with the user breathing interface and provide an output signal indicative of the pressure of the supply of breathing gas at the user breathing interface. A measurement lumen may be resident within the breathing gas tube, which measurement lumen fluidly communicates the user breathing interface with the pressure sensor.

A controller which couples the measurement lumen to the blower controls the output of the breathing gas from the blower. In a preferred embodiment, the controller maintains a pressure of the breathing gas at the user breathing interface to between about 1 cm H₂0 to about 6 cm H₂0. Also in the preferred embodiment, the blower assembly accelerates the breathing gas at about 1.5 l/min/s over a flow rate of about 0 l/min to about 100 l/min. The breathing gas supply tube may have a cross-sectional area in the range of 0.5 mm² to 2.0 mm². Other sizes, dimensions and configurations of flow parameters are contemplated to be within the scope of the invention disclosed herein.

The blower includes a blower motor and an impeller. A speed of the motor may be controlled based upon, at least in part, the control signal communicated through the sensing lumen. In this way, the pressure of breathing gas supplied to the user breathing interface may be closely controlled to within predetermined parameters specified for the particular user.

Referring now to the drawings, in a first embodiment of the invention as shown in FIGS. 1-6, the breathing gas tube 10 includes an asymmetrically-shaped outer wall member 20, which defines a hollow interior capable of conveying any type of fluid, and a coextruded inner strengthening rib or partition wall 30. Rib 30 divides tube 10 into first and second flow chambers 40, 50, respectively. Rib 30 has, in the preferred embodiment, an upper wall member 32 and a lower wall section 36 which defines and surrounds a third, pressure sensing, lumen 60. Pressure lumen 60 may have any cross-sectional configuration desirable, the circular cross-section shown being merely exemplary. Alternatively, pressure lumen 60 may be dispensed with without departing from the spirit of the invention. In the event that there is no pressure lumen such as third lumen 60, rib 30 may extend from the upper wall section to the lower wall section as a rib having a constant cross-sectional area, or the cross-sectional area may be varied depending upon the bending-resistance characteristics desired for tube 10. For example, the increased amount of material used to created third lumen 60 causes a commensurate strengthening of the tube 10 in one or more directions.

Chambers 40 and 50 are adapted to convey breathing gas from the blower to the user breathing interface, and are sometimes herein referred to as the “first” and “second” lumens. Third lumen 60 fluidly communicates the above-referenced pressure sensor with a desired location at which pressure (or one or more other parameters) is to be measured, such as within the user breathing interface.

Outer wall 20 may be fabricated from a resinous or polymeric material such as medical grade PVC in the preferred embodiment. In the preferred embodiment, the material out of which external wall 20 is fabricated has a durometer of between 40 Shore A and 70 Shore A, and may be 50 Shore A. Also in the preferred embodiment, rib 30 is also preferably fabricated of medical grade PVC, and preferably has a durometer of between 40 Shore A and 80 Shore A, and may be 70 Shore A.

It is preferred to select the material out of which outer wall 20 and rib 30 are constructed from the same family of materials so that when they are coextruded a secure bond will be formed there between due to the molecular compatibility of the material.

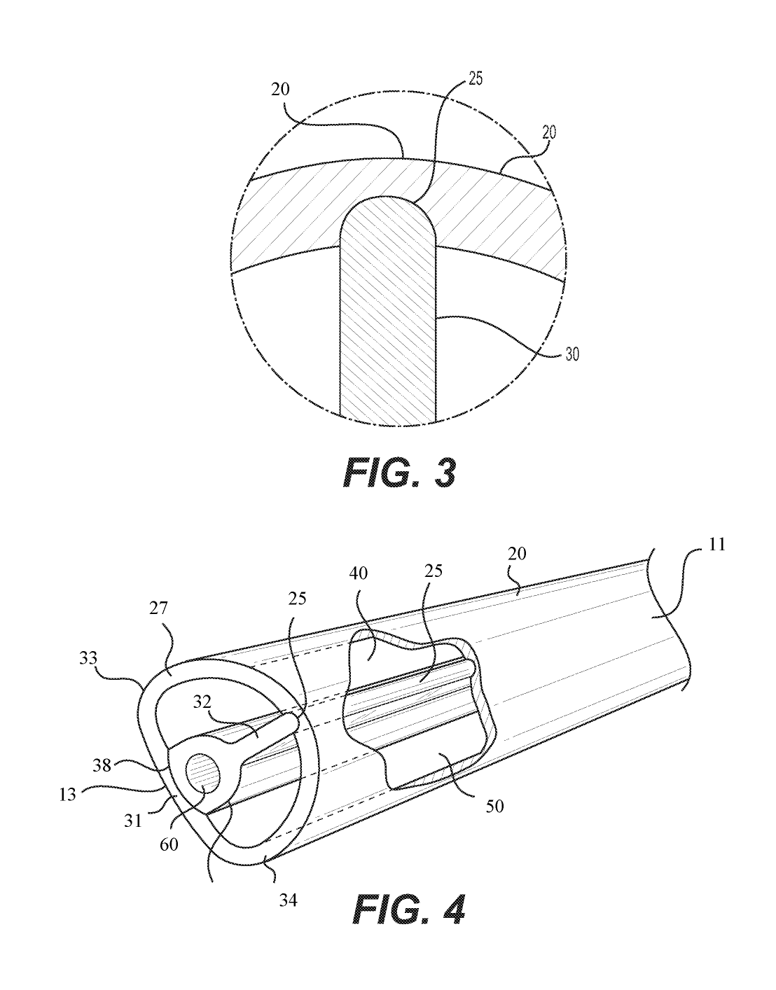

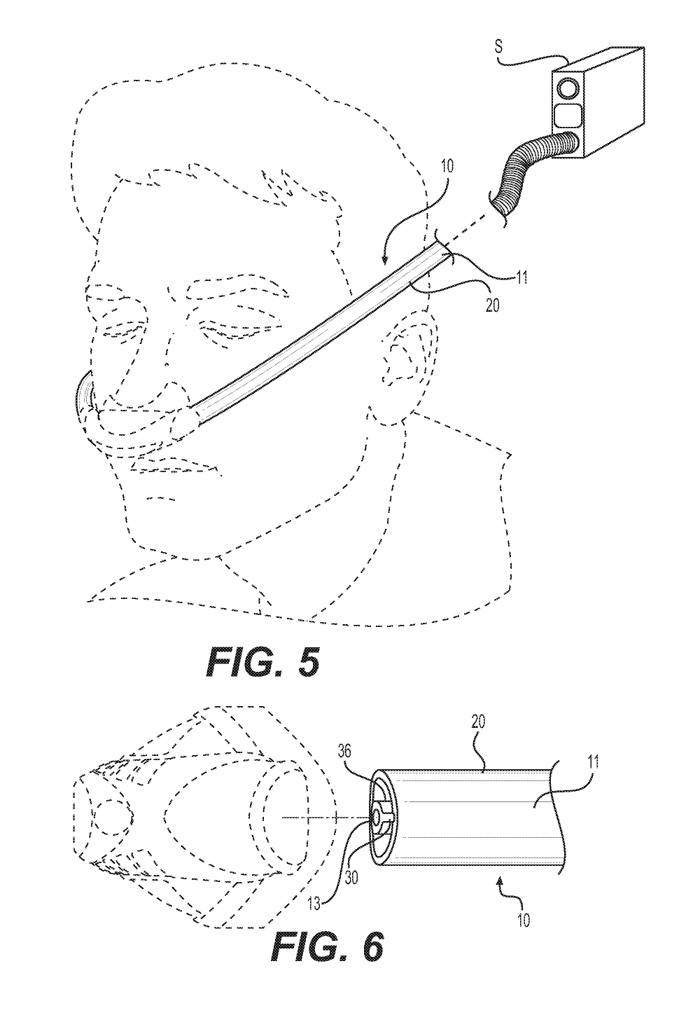

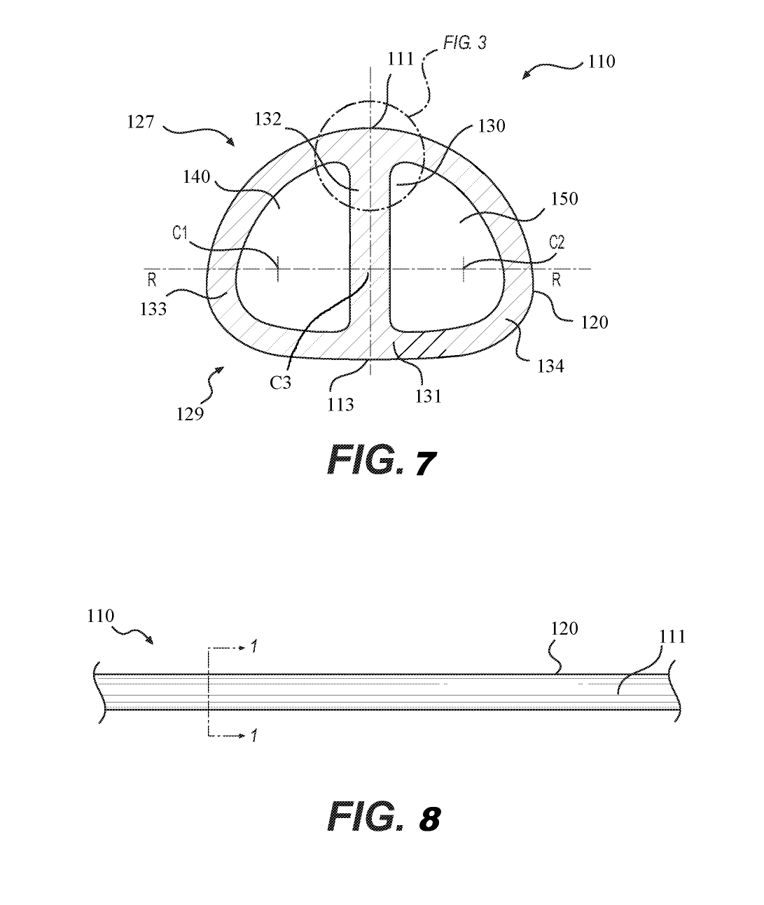

The bond area 25 between upper rib wall 32 and outer wall 20 is preferably formed in the shape of the arch shown in FIGS. 1 and 3. A preferred radius of bond area 25 is approximately 0.025 inches, and it is preferred that rib 30 extend in the range of halfway through/into tube wall 20.

Bond area 38, where lower section 36 of rib 30 mates with wall 20, is preferably shaped in the manner shown in FIG. 1. Preferably, bond area 35 has a length of approximately in the range of 0.166 inches and extends in the range of approximately 0.020 inches into wall 20.

Wall 20 is defined by an upper section 27, that is, substantially the portion of wall 20 above reference line R. A lower portion 29 of outer wall 20 is that portion of said wall 20 lying below reference line R. Upper section 27 has a radius of curvature centered about center point C₃ of preferably in the range of 0.240 inches, and is substantially bisected by rib 30 in the preferred embodiment.

Lower portion or section 29 of wall 20 has a complex curvature in the preferred embodiment. In one embodiment, a central section, or second wall segment, 31 thereof, which is substantially bisected by lower portion 36 of rib 30, has a radius of curvature of approximately 0.794 inches. On opposed sides of lower wall section 31 are first and third curved wall segments 33, 34. Preferably, the curvature of each of lower section wall segments 33, 34 are similarly shaped, in the preferred embodiment having a radius of curvature of approximately 0.105 inches. First and third curved segments 33, 34 of lower section 29 of outer wall 20 preferably are preferably centered about respective centers C₁ and C₂.

As stated earlier, rib 30 and outer wall 20 are, preferably, coextruded. However, any other manner of assembling outer wall 20 and inner strengthening rib 30 is contemplated to be within the scope of this invention, including extruding them simultaneously when they are fabricated of material having a single durometer.

The intersection of upper rib wall 32 with lower rib, or partition, section 36 may employ rounded interior corners 70, 71 to facilitate a low friction environment for the fluid passing through chambers 40, 50. Preferably, the radius of corners 70, 71 is in the range of approximately 0.020 inches.

Third lumen 60 preferably has an inner diameter of approximately 0.079 inches. The outer diameter of lower rib section 36 is preferably, but not by way of limitation, approximately in the range of 0.169 inches. The overall height of tube 10 when viewed in the orientation shown in FIG. 1 is approximately in the range of 0.344 inches, while the overall width thereof is preferably in the range of approximately 0.478 inches.

The upper wall 32 of rib 30 may or may not be fluid-permeable, such that, if the fluid to be conveyed in chambers 40 and 50 is the same, there need be no fluid-tight barrier between them. By constructing upper wall 32 of fluid-permeable material, the fluid in chambers 40 and 50 can intermix. Therefore, if the hose 10 is bent to such an extent that one of chambers 40 or 50 are occluded, there can still be essentially unimpeded fluid flow in the remainder of tube 10. One manner in which to render upper wall 32 fluid-permeable is to create pores therein. Another manner in which to render upper wall 32 fluid-permeable is to otherwise create apertures therein

It is to be understood that the connections between rib 30 and outer wall 20 may take any suitable form. All that is required is that rib 30 and outer wall 20 are attached such that they move together when forces are imposed upon them. The bond between rib 30 and outer tube 20 need not involve penetration into the outer tube wall by rib 30.

In the case of a device for the treatment of snoring, it is preferred that the pressure in the user breathing interface be fed back to the controller to regulate the flow rate and/or pressure of the breathing gas delivered through tube 10. However, third lumen 60 need not be included, such that rib 30 may extend as an elongated wall, having a constant or varying cross-section, disposed substantially across the center, or in any other orientation, within outer wall 20. In many CPAP devices, pressure is sensed not at the user breathing interface, but at or near the blower apparatus. In such apparatus, a sensing, or third, lumen such as lumen 60 may be unnecessary. Nevertheless, it is desirable in many applications to use a hose having a “flattened” side (e.g., D-shaped) such as tube 10, resulting in an asymmetrical cross-section, such that the principals upon which the instant invention are founded apply regardless of whether or not a third lumen is provided.

In general, the invention is directed to a fluid delivery hose or tube which has an asymmetrical cross-section but which exhibits essentially symmetrical bending resistance in all directions. This is accomplished by the selection and arrangement of materials and structural dimensions of the tube 10, the preferred embodiment of which is disclosed herein.

In the preferred embodiment, therefore, the outer hose forms a substantially D-shaped cross-section defined by an upper section 27 which has a substantially constant radius of curvature that extends from the point that wall 20 intersects reference line R on the left side of tube 10 to the point that wall 20 intersects reference line R on the right side of tube 10. In the preferred embodiment, the distance from center C₃ to the uppermost point 11 of tube 10 is approximately 0.224 inches. The distance from center C₃ to the lowermost point 13 of the lower section 29 of wall 20 is approximately 0.12 inches. The uppermost and lowermost points 11 and 13 correspond to uppermost and lowermost edges 11 and 13 of outer tube 20. In this embodiment, a strengthening rib 30 is interposed between the upper section 27 of wall 20 and the lower section 29 of wall 20. Rib 30 is preferably coextruded with wall 20 and is preferably fabricated out of a material having a durometer that is equal to or higher than the durometer of the material out of which wall 20 is manufactured. It has been determined that configuring the tube 10 in this manner results in a surprising, unexpected result, where the tube exhibits uniform bending moment of inertia balance in all directions. In the preferred embodiment, the combination of the structural shapes of the walls 20 and 30, and/or the disclosed durometer ranges, yields results never before achieved in an asymmetrically shaped tube.

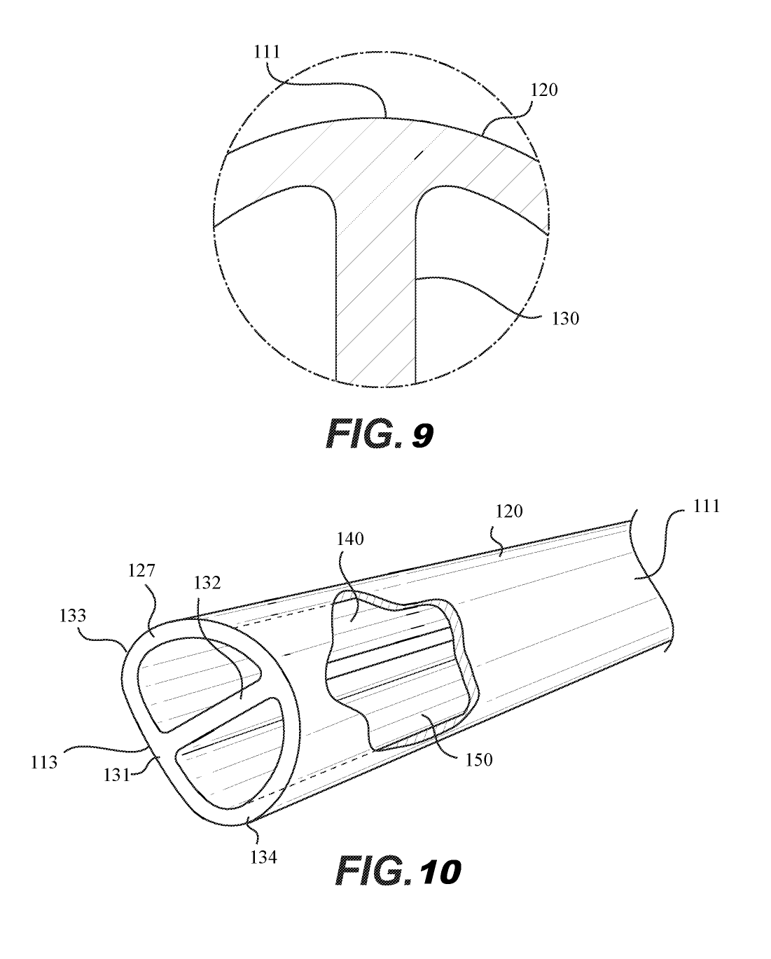

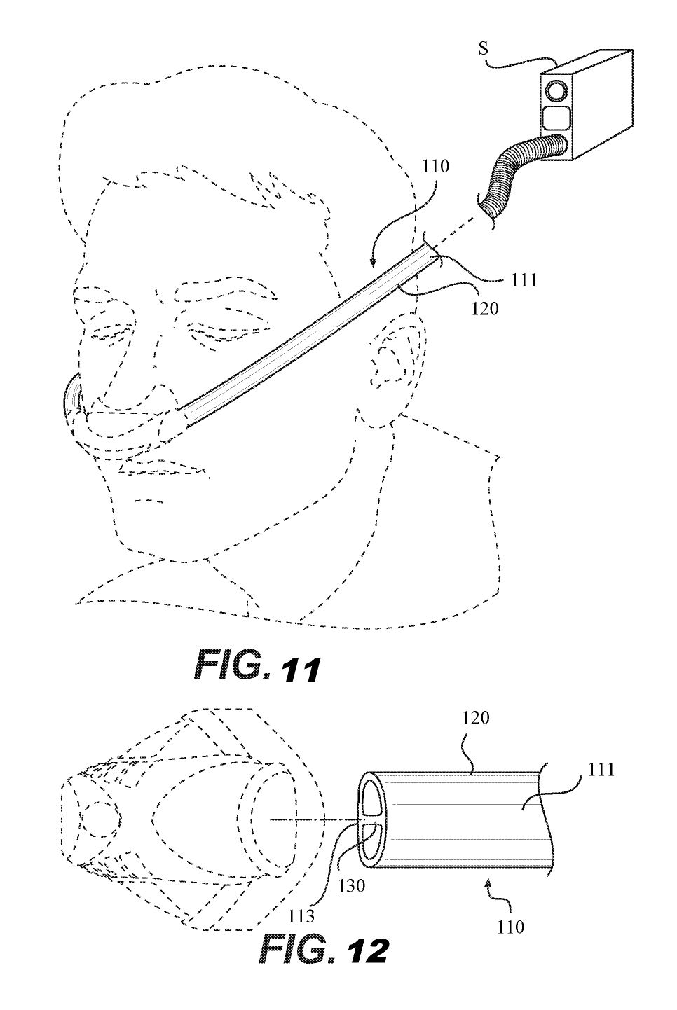

Referring now to FIGS. 7-12, in a second embodiment of the invention, the breathing gas tube 110 includes an asymmetrically-shaped outer wall member 120, which defines a hollow interior capable of conveying any type of fluid, and a coextruded inner strengthening rib or partition wall 130. Rib 130 divides tube 110 into first and second flow chambers 140, 150, respectively. Rib 130 has, in the preferred embodiment, a wall member 132 and rib 130 may have a constant cross-sectional area, or the cross-sectional area may be varied depending upon the bending-resistance characteristics desired for tube 110.

Chambers 140 and 150 are adapted to convey breathing gas from the blower to the user breathing interface, and are sometimes herein referred to as the “first” and “second” lumens.

Outer wall 120 may be fabricated from a resinous or polymeric material such as medical grade PVC in the preferred embodiment. In the preferred embodiment, the material out of which external wall 120 is fabricated has a durometer of between 40 Shore A and 70 Shore A, and may be 50 Shore A. Also in the preferred embodiment, rib 30 is also preferably fabricated of medical grade PVC, and preferably has a durometer of between 40 Shore A and 80 Shore A, and may be 70 Shore A.

It is preferred to select the material out of which outer wall 120 and rib 130 are constructed from the same family of materials so that when they are coextruded a secure bond will be formed there between due to the molecular compatibility of the material.

Wall 120 is defined by an upper section 127, that is, substantially the portion of wall 120 above reference line R. A lower portion 129 of outer wall 120 is that portion of said wall 120 lying below reference line R. Upper section 127 has a radius of curvature centered about center point C₃ of preferably in the range of 0.240 inches, and is substantially bisected by rib 130 in the preferred embodiment.

Lower portion or section 129 of wall 120 has a complex curvature in the preferred embodiment. In one embodiment, a central section, or second wall segment, 131 thereof, which is substantially bisected by rib 130, has a radius of curvature of approximately 0.794 inches. On opposed sides of lower wall section 131 are first and third curved wall segments 133, 134. Preferably, the curvature of each of lower section wall segments 133, 134 are similarly shaped, in the preferred embodiment having a radius of curvature of approximately 0.105 inches. First and third curved segments 133, 134 of lower section 129 of outer wall 120 preferably are preferably centered about respective centers C₁ and C₂.

As stated earlier, rib 130 and outer wall 120 are, preferably, coextruded. However, any other manner of assembling outer wall 120 and inner strengthening rib 130 is contemplated to be within the scope of this invention, including extruding them simultaneously when they are fabricated of material having a single durometer.

The overall height of tube 110 when viewed in the orientation shown in FIG. 7 is approximately in the range of 0.344 inches, while the overall width thereof is preferably in the range of approximately 0.478 inches.

The wall 132 of rib 130 may or may not be fluid-permeable, such that, if the fluid to be conveyed in chambers 140 and 150 is the same, there need be no fluid-tight barrier between them. By constructing wall 132 of fluid-permeable material, the fluid in chambers 140 and 150 can intermix. Therefore, if the hose 110 is bent to such an extent that one of chambers 140 or 150 are occluded, there can still be essentially unimpeded fluid flow in the remainder of tube 110. One manner in which to render wall 132 fluid-permeable is to create pores therein. Another manner in which to render wall 132 fluid-permeable is to otherwise create apertures therein

It is to be understood that the connections between rib 130 and outer wall 120 may take any suitable form. All that is required is that rib 130 and outer wall 120 are attached such that they move together when forces are imposed upon them. The bond between rib 130 and outer tube 120 need not involve penetration into the outer tube wall by rib 130.

In general, the invention is directed to a fluid delivery hose or tube which has an asymmetrical cross-section but which exhibits essentially symmetrical bending resistance in all directions. This is accomplished by the selection and arrangement of materials and structural dimensions of the tube 110, the preferred embodiment of which is disclosed herein.

In the preferred embodiment, therefore, the outer hose forms a substantially D-shaped cross-section defined by an upper section 127 which has a substantially constant radius of curvature that extends from the point that wall 120 intersects reference line R on the left side of tube 110 to the point that wall 120 intersects reference line R on the right side of tube 110. In the preferred embodiment, the distance from center C₃ to the uppermost point 111 of tube 110 is approximately 0.224 inches. The distance from center C₃ to the lowermost point 113 of the lower section 129 of wall 120 is approximately 0.12 inches. The uppermost and lowermost points 111 and 113 correspond to uppermost and lowermost edges 111 and 113 of outer tube 120. In this embodiment, a strengthening rib 130 is interposed between the upper section 127 of wall 120 and the lower section 129 of wall 120. Rib 130 is preferably coextruded with wall 120 and is preferably fabricated out of a material having a durometer that is equal to or higher than the durometer of the material out of which wall 120 is manufactured. It has been determined that configuring the tube 110 in this manner results in a surprising, unexpected result, where the tube exhibits uniform bending moment of inertia balance in all directions. In the preferred embodiment, the combination of the structural shapes of the walls 120 and 130, and/or the disclosed durometer ranges, yields results never before achieved in an asymmetrically shaped tube.

While the invention has been described in its preferred form or embodiment with some degree of particularity, it is understood that this description has been given only by way of example and that numerous changes in the details of construction, fabrication, and use, including the combination of structural arrangement and sizes of features, may be made without departing from the spirit and scope of the invention.

What is claimed is:

1. A hose adapted to carry one or more fluids, the hose exhibiting substantially omnidirectional resistance to bending and collapse, comprising:
an outer wall defining an interior volume adapted to carry one or more fluids, the outer wall comprised of an upper section and a lower section;
an inner strengthening rib having a length which extends substantially an entire length of said interior volume, a first end of the rib being connected to the upper section of the outer wall, a second end of said rib being connected to the lower section of the outer wall;
the upper section of the outer wall having a substantially constant radius of curvature, the lower section of the outer wall being comprised of first, second and third segments, the first and third segments being separated from one another by the second segment;
the first and third segments of the lower section of the outer wall having substantially the same radius of curvature as each other, the radius of curvature of the first and third segments being less than a radius of curvature of the upper section, the second segment of the lower section of the outer wall having a radius of curvature that is larger than the radius of curvature of the upper section of the outer wall;
wherein the hose exhibits a substantially omnidirectional bending moment of inertia; wherein the inner strengthening rib divides the hose into two lumens that are substantially equal in cross-sectional area.

2. The hose of claim 1, wherein the outer wall is manufactured from a polymeric material having a durometer in the range of 40 Shore A and 70 Shore A.

3. The hose of claim 1, wherein the inner rib is manufactured from a polymeric material having a durometer in the range of 40 Shore A and 80 Shore A.

4. The hose of claim 1, wherein the outer wall is manufactured from a polymeric material having a durometer of approximately 50 Shore A.

5. The hose of claim 1, wherein the inner rib is manufactured from a polymeric material having a durometer of approximately 70 Shore A.

6. The hose of claim 1, wherein the outer wall is manufactured from a polymeric material having a durometer in the range of 40 Shore A and 70 Shore A, and wherein the inner rib is manufactured from a polymeric material having a durometer in the range of 40 Shore A and 80 Shore A.

7. The hose of claim 1, wherein the outer wall is manufactured from a polymeric material having a durometer of approximately 50 Shore A, and wherein the inner rib is manufactured from a polymeric material having a durometer of approximately 70 Shore A.

8. An extruded polymer hose, the hose comprising:
at least one outer tube having an outer tube durometer forming two lumens, said outer tube defined by an upper section and a lower section, with at least a portion of said lower section having a flattened contour, said outer tube being asymmetric about a plane passing through a central axis of the tube, which plane is parallel to both an uppermost edge of the tube and a lowermost edge of the tube.

9. The tube of claim 8 wherein said upper section of said outer polymer tube has a radius of curvature of 0.240 inches.

10. The tube of claim 8 wherein said outer tube is D-shaped.

11. The tube of claim 9 wherein said outer tube is D-shaped.

12. The tube of claim 8 wherein said outer tube is fabricated from a resinous material such as medical grade PVC.

13. The tube of claim 9 wherein said outer tube is fabricated from a resinous material such as medical grade PVC.

14. The tube of claim 10 wherein said outer tube is fabricated from a resinous material such as medical grade PVC.

15. The tube of claim 8 wherein said outer tube durometer is between 40 Shore A and 70 Shore A.

16. The tube of claim 9 wherein said outer tube durometer is between 40 Shore A and 70 Shore A.

17. The tube of claim 10 wherein said outer tube durometer is between 40 Shore A and 70 Shore A.

18. The tube of claim 11 wherein said outer tube durometer is between 40 Shore A and 70 Shore A.

19. The tube of claim 8, further comprising at least one co-extruded inner polymer rib disposed within said outer tube, said rib substantially bisecting the outer tube, and said rib having a rib durometer.

20. The tube of claim 19, wherein said rib is fabricated of medical grade PVC.

21. The tube of claim 19, wherein said rib durometer is between 40 Shore A and 80 Shore A.

22. The tube of claim 19, wherein said rib defines at least one co-extruded inner lumen.

23. The tube of claim 9, wherein the upper wall member of the rib is bonded to said upper section of the outer tube.

24. The tube of claim 19 wherein the rib extends into said upper section of the outer tube.

25. The tube of claim 19, wherein said rib durometer and said inner tube durometer are the same.

26. The tube of claim 8, wherein a height of said outer tube, as measured from a bottom edge of the tube to a top edge of the tube, is 0.344 inches, and also wherein a width of the outer tube is 0.478 inches.

27. The tube of claim 19, wherein a height of said outer tube, as measured from a bottom edge of the tube to a top edge of the tube, is 0.344 inches, and also wherein a width of the outer tube is 0.478 inches.

28. An extruded polymer tube, the tube comprising:
at least one extruded outer tube having an outer tube durometer forming at least a first lumen, said outer tube defined by an upper section and a lower section, with at least a portion of said lower section having a flattened contour, said outer tube being asymmetric about a plane passing through a central axis of the tube, which plane is parallel to both an uppermost edge of the tube and a lowermost edge of the tube; and
at least one co-extruded inner rib disposed within said outer tube, said rib substantially bisecting the outer tube, and said rib having a rib durometer, wherein said rib durometer is greater than or equal to the tube durometer;
wherein the inner rib defines two lumens.

29. The tube of claim 28, wherein said rib and said outer tube are fabricated from a similar family of materials to promote bonding during the co-extrusion of said outer tube and said rib, wherein said materials are medical grade PVC.

30. A system for treating sleep disordered breathing, comprising:
a source of pressurized air;
a hose adapted to carry one or more fluids, the hose comprising:
an outer wall defining an interior volume adapted to carry one or more fluids, the outer wall comprised of an upper section and a lower section;
an inner strengthening rib having a length which extends within at least a portion of a length of said interior volume, a first end of the rib being connected to the upper section of the outer wall, a second end of said rib being connected to the lower section of the outer wall;
the upper section of the outer wall having a substantially constant radius of curvature, the lower section of the outer wall being comprised of first, second and third segments, the first and third segments being separated from one another by the second segment;
the first and third segments of the lower section of the outer wall having substantially the same radius of curvature as each other, the radius of curvature of the first and third segments being less than a radius of curvature of the upper section, the second segment having a radius of curvature that is larger than the radius of curvature of the upper section of the outer wall;
wherein the hose exhibits a substantially omnidirectional bending moment of inertia;
a respiratory interface, the hose fluidly communicating the source of pressurized air with the nasal interface;
wherein the inner rib defines two lumens which extend along substantially the entire length of the rib.

31. The hose of claim 30, wherein the outer wall is manufactured from a polymeric material having a durometer in the range of 40 Shore A and 70 Shore A.

32. The hose of claim 30, wherein the inner rib is manufactured from a polymeric material having a durometer in the range of 40 Shore A and 80 Shore A.

33. The hose of claim 30, wherein the outer wall is manufactured from a polymeric material having a durometer of approximately 50 Shore A.

34. The hose of claim 30, wherein the inner rib is manufactured from a polymeric material having a durometer of approximately 70 Shore A.

35. The hose of claim 30, wherein the outer wall is manufactured from a polymeric material having a durometer in the range of 40 Shore A and 70 Shore A, and wherein the inner rib is manufactured from a polymeric material having a durometer in the range of 40 Shore A and 80 Shore A.

36. The hose of claim 30, wherein the outer wall is manufactured from a polymeric material having a durometer of approximately 50 Shore A, and wherein the inner rib is manufactured from a polymeric material having a durometer of approximately 70 Shore A.
